(19) United States
(12) Patent Application Publication
(10) Pub. No.: US 2026/0076831 A1
(43) Pub. Date: Mar. 19, 2026

(54) TREATMENT APPARATUS AND METHOD FOR PROVIDING CONTROL DATA FOR AN OPHTHALMOLOGICAL LASER OF A TREATMENT APPARATUS FOR THE LASER-INDUCED REFRACTIVE INDEX CHANGE

(71) Applicant: SCHWIND eye-tech-solutions GmbH, Kleinostheim (DE)

(72) Inventors: Pascal NAUBEREIT, Laufach (DE); Samuel ARBA MOSQUERA, Aschaffenburg (DE)

(21) Appl. No.: 18/889,738

(22) Filed: Sep. 19, 2024

(51) Int. Cl. A61F 9/008

(52) U.S. Cl. CPC A61F 9/008; A61F 2009/00872

(57) ABSTRACT

The invention relates to a treatment apparatus (10) and a method for providing control data for an ophthalmological laser (12) of a treatment apparatus (10) for the laser-induced refractive index change. As steps, the method includes determining (S10) treatment positions (16) in a cornea (14) of an eye, which are intended for the change by the laser-induced refractive index change; determining (S12) an orientation of collagen fibers (30) to be expected in the cornea (14) in the respective treatment positions (16) based on collagen fiber data of the cornea (14); setting (S14) a polarization property (24, 26) of a laser beam (20) of the laser (12) based on the orientation of the collagen fibers (30) to be expected for a respective treatment position (30); and providing (S16) the control data, which includes the set polarization property (24, 26) for the respective treatment position (16).

### FIELD

The invention relates to a method for providing control data for an ophthalmological laser in a treatment apparatus for laser-induced refractive index change. Furthermore, the invention relates to a control device, which is configured to perform the method, to a computer program comprising commands, which cause the control device to execute the method, and to a computer-readable medium, on which such a computer program is stored.

### BACKGROUND

Treatment apparatuses and methods for controlling ophthalmological lasers for correcting an optical visual disorder and/or pathologically or unnaturally altered areas of the cornea are known in the prior art. Therein, pulsed lasers and a beam focusing device can, for example, be formed such that laser pulses effect a photodisruption and/or ablation in a focus situated within the organic tissue to remove a tissue, in particular a tissue lenticule, from the cornea.

Besides the previously mentioned possibilities, the laser-induced optical refractive index change (LIRIC) is a further possibility of treating visual disorders. Herein, a tissue structure is changed by irradiation of laser pulses with short laser pulse length without generating an optical breakdown, whereby a refractive index of the concerned tissue structures changes and thus wavefronts can be adapted for correcting the visual disorder. In the laser-induced refractive index change, a separation of monomer compounds of collagen fibers in the cornea is in particular effected, such that a collagen matrix of the collagen fibers modifies and densifies. Hereby, a refractive index increases, whereby structures can thus be generated in the cornea to shape an optical vision.

### SUMMARY

It is the object of the invention to improve a laser-induced optical refractive index change.

This object is solved by the examples provided herein. Advantageous embodiments of the invention are disclosed in the dependent claims, the following description as well as the figures.

The invention is based on the idea that a specific collagen fiber arrangement, which is present within the cornea, is taken into account in the irradiation by laser pulses. Herein, the collagen fiber arrangement can vary depending on a depth in the cornea. For example, a diffuse or chaotic arrangement of collagen fibers can be present in anterior corneal layers, and they can be provided more orderly, in particular parallel to each other, in posterior layers. In order to improve the effect of the laser-induced refractive index change, it can therefore be provided that a polarization direction of the laser pulses is adapted to the orientation of the collagen fibers.

An aspect of the invention relates to a method for providing control data for an ophthalmological laser of a treatment apparatus for the laser-induced refractive index change, wherein the method comprises the following steps performed by a control device. An appliance or an appliance component, in particular a processor or microprocessor, is to be understood by a control device, by which the following steps can be autonomously or partially autonomously performed: determining treatment positions in a cornea of an eye, which are intended for the change by the laser-induced refractive index change; determining an orientation of collagen fibers to be expected in the cornea in the respective irradiation positions based on collagen fiber data of the cornea; setting a polarization property of a laser beam of the laser based on the orientation of the collagen fibers to be expected for a respective treatment position; and providing the control data, which includes the set polarization property for the respective treatment position, are effected.

In other words, treatment positions in a cornea can first be determined or preset, in which a laser-induced refractive index change is to occur, for example to generate a preset structure in the cornea to correct a visual disorder. Herein, the treatment positions can be coordinates in the cornea, which are to be irradiated with laser pulses for the refractive index change.

Then, it can be determined for each treatment position, which orientation of the collagen fibers is to be expected in the respective treatment position. The orientation of the collagen fibers in the cornea can be predetermined and be presented to the control device as collagen fiber data. For example, the collagen fiber data can include measurement data of one or more corneas. For example, the eye to be treated can be measured by diagnostic examinations to ascertain the orientation of the collagen fibers. Alternatively, data of other eyes or corneas can be present, in which the orientation of collagen fibers has already been determined, wherein this orientation can be adopted for the eye to be treated. Hereto, the orientations of the collagen fibers can, for example, originate from histological examinations of one or more eyes.

When the orientation of the collagen fibers to be expected has been determined for the respective treatment position, a polarization property of the laser beam can be set for the treatment position depending on the orientation. In order to adjust the polarization property in the treatment, a polarization device of the treatment apparatus can, for example, be controlled by the control data. For example, the polarization device can comprise one or more retardation plates to adjust the polarization property. The laser beam can, in particular, be linearly or circularly polarized as the polarization property.

Finally, control data for controlling the ophthalmological laser can be provided, which includes the set polarization property for the respective treatment position. In particular, the ophthalmological laser can subsequently be controlled by the control data. The control data can include a respective dataset for positioning and/or for focusing individual laser pulses in the cornea. Additionally or alternatively, a respective dataset for adjusting at least one beam device for beam guidance and/or beam shaping and/or beam deflection and/or beam focusing and/or polarization adjustment of a laser beam of the respective laser can be included in the control data.

By the invention, the advantage arises that the laser beam can be polarized corresponding to the orientation of the collagen fibers, whereby an effect of the laser-induced refractive index change can be intensified. Thus, the laser-induced refractive index change can overall be improved.

The invention also includes embodiments, by which additional advantages arise.

In an embodiment the orientation of the collagen fibers to be expected is determined based on a radial position and/or a depth of the treatment position in the cornea. In other words, the orientation of the collagen fibers can be recorded in the collagen fiber data depending on the radial position and/or the depth of the treatment position. Herein, the collagen fiber data can for example provide a mathematical model or a look-up table to determine the orientation of the collagen fibers to be expected in the respective treatment position.

In another embodiment a circularly polarized laser beam and/or a linearly polarized laser beam comprising a polarization angle are adjusted as the polarization property. In the circular polarization, the polarization direction can change with constant angular speed, wherein this polarization property is preferred in areas in which a disorderly orientation of collagen fibers is to be expected. The linear polarization, in which the polarization direction or a polarization angle is constant, can in particular be used in an orderly arrangement of the collagen fibers, such that the polarization is corresponding to the orientation of the collagen fibers, to achieve an increased laser-induced refractive index change effect.

In another embodiment the collagen fiber data is predetermined by a histological analysis of multiple corneas. Herein, a morphological or histological examination can be performed within the scope of an anatomic dissection of corneas, to ascertain the orientation of collagen fibers. This means, it can be ascertained how the orientation of the collagen fibers is usually present in human and/or animal eyes, to thus obtain an assumption about the orientation of the collagen fibers in each eye.

In another embodiment, the collagen fiber data is predetermined by measurements of the cornea of the eye. This means that preliminary examinations can be performed for the eye to be treated, from which the orientation of the collagen fibers is determined. For example, this can be performed by microscopic examinations on the eye to be treated. Hereby, the advantage arises that the orientation of the collagen fibers can be provided individually for the patient, whereby an accuracy in the adjustment of the polarization property can be increased.

In another embodiment an orientation of the collagen fibers is assumed to be diffuse above a preset corneal depth and to be parallel below the preset corneal depth, wherein a circular polarization is set as the polarization property of the laser beam for treatment positions above the preset corneal depth and a linear polarization below the preset corneal depth. This means that a diffuse arrangement of collagen fibers can be present in an anterior area of the cornea, which is above the preset corneal depth. In order to increase an efficiency in the irradiation of this diffuse arrangement for the laser-induced refractive index change, it can therefore be advantageous to use a circularly polarized laser beam. In contrast, in a posterior area of the cornea, which is below the preset corneal depth, the collagen fibers can be orderly oriented, in particular parallel to each other. In this area, the use of linearly polarized light is advantageous to achieve an improved laser-induced refractive index change effect. Herein, the preset corneal depth can be preset, in particular depending on a sex, an age and/or an ethnicity of a patient. For example, the preset corneal depth can be at 150 micrometers within the stroma, wherein further corneal depths can also be provided. By this embodiment, the advantage arises that a suitable polarization property can be provided for each depth in the cornea.

In another embodiment a progression of the collagen fibers below the preset corneal depth is assumed to be hyperbolic viewed in radial direction, wherein a polarization angle of the linear polarization is adjusted depending on the hyperbolic progression as the polarization property. This means that the polarization direction can be co-rotated depending on the treatment position and the hyperbolic progression. Herein, it is meant by hyperbolic that the collagen fibers can extend from the outside towards a center of the cornea and subsequently again to the outside. Herein, the cornea can be divided into multiple quadrants, which are point-symmetric to each other, wherein the collagen fibers can have such a hyperbolic progression in each quadrant, which is, for example, illustrated in FIG. 2.

A further aspect relates to a method for controlling a treatment apparatus. Therein, the method includes the method steps of at least one form of configuration of the method as it was previously described. Furthermore, the method for controlling the treatment apparatus also includes the step of transferring the provided control data to at least one ophthalmological laser of the treatment apparatus and controlling the treatment apparatus and/or the laser with the control data.

The respective method can include at least one additional step, which is executed if and only if an application case or an application situation occurs, which has not been explicitly described here. For example, the step can include the output of an error message and/or the output of a request for inputting a user feedback. Additionally or alternatively, it can be provided that a default setting and/or a predetermined initial state are adjusted.

A further aspect of the invention relates to a control device, which is configured to perform the steps of at least one embodiment of one or both of the previously described methods. Thereto, the control device can comprise a computing unit for electronic data processing such as, for example, a processor. The computing unit can include at least one microcontroller and/or at least one microprocessor. The computing unit can be configured as an integrated circuit and/or microchip. Furthermore, the control device can include an (electronic) data memory or a storage unit. A program code can be stored on the data memory, by which the steps of the respective embodiment of the respective method are encoded. The program code can include the control data for the respective laser. The program code can be executed by the computing unit, whereby the control device is caused to execute the respective embodiment. The control device can be formed as a control chip or control unit. The control device can for example be encompassed by a computer or computer cluster

A further aspect of the invention relates to a computer program. The computer program includes commands, which for example form a program code. The program code can include at least one control dataset with the respective control data for the respective laser. Upon execution of the program code by a computer or a computer cluster, it is caused to execute the previously described method or at least one embodiment thereof.

A further aspect of the invention relates to a computer-readable medium (storage medium), on which the above mentioned computer program and the commands thereof, respectively, are stored. For executing the computer program, a computer or a computer cluster can access the computer-readable medium and read out the content thereof. The storage medium is for example formed as a data memory, in particular at least partially as a volatile or a non-volatile data memory. A non-volatile data memory can be a flash memory and/or an SSD (solid state drive) and/or a hard disk. A volatile data memory can be a RAM (random access memory). For example, the commands can be present as a source code of a programming language and/or as assembler and/or as a binary code.

A further aspect of the invention relates to a treatment apparatus comprising at least one ophthalmologic laser for the laser-induced refractive index change of a cornea of a human or animal eye. The treatment apparatus further comprises a control device and a polarization device, wherein the polarization device is configured to adjust a polarization property of a laser beam of the ophthalmologic laser, wherein the control device is configured to determine an expected orientation of collagen fibers in the cornea at predetermined treatment positions in the cornea of the eye that are intended to be changed by the laser-induced refractive index change on the basis of predetermined collagen fiber data of the cornea, and the polarization device is configured to adjust the polarization property depending on the expected orientation of the collagen fibers at the respective treatment position during irradiation with the ophthalmologic laser.

In particular, the polarization device of the treatment apparatus may comprise at least one waveplate and/or polarization filter in order to set and/or change the polarization property of the laser beam. For example, the polarization device may polarize the laser beam circularly and/or linearly, whereby preferably a polarization angle may be set.

In an embodiment of the treatment apparatus the polarization device comprises one or more waveplates, in particular an arrangement of waveplates, and/or one or more rotatable polarizers. The waveplate may be, for example, λ/2 and/or λ/4 plates that retard light waves so that a polarization direction may be rotated and/or a switch between linearly and circularly polarized light is achieved. Alternatively or additionally, at least one polarizer may be provided, in particular a polarization filter, which is rotatably mounted, whereby a rotation may be set by activation by the control device. This allows a polarization angle to be set. The polarizer may be configured as a linear polarizer and/or comprise a circular polarizer.

Further features and advantages of one of the described aspects of the invention can result from the embodiments of another one of the aspects of the invention. Thus, the features of the embodiments of the invention can be present in any combination with each other if they have not been explicitly described as mutually exclusive.

### BRIEF DESCRIPTION OF THE DRAWINGS

In the following, additional features and advantages of the invention are described in the form of advantageous execution examples based on the figure(s). The features or feature combinations of the execution examples described in the following can be present in any combination with each other and/or the features of the embodiments. This means, the features of the execution examples can supplement and/or replace the features of the embodiments and vice versa. Thus, embodiments are also to be regarded as encompassed and disclosed by the invention, which are not explicitly shown or explained in the figures, but arise from and can be generated by separated feature combinations from the execution examples and/or embodiments. Thus, embodiments are also to be regarded as disclosed, which do not comprise all of the features of an originally formulated claim or extend beyond or deviate from the feature combinations set forth in the relations of the claims. To the execution examples, there shows:

FIG. 1 depicts a treatment apparatus with an ophthalmological laser according to an exemplary embodiment.

FIG. 2 depicts a schematic representation of a progression of collagen fibers in a corneal plane.

FIG. 3 depicts a schematic method diagram for providing control data.

### DETAILED DESCRIPTION

In the figures, identical or functionally identical elements are provided with the same reference characters.

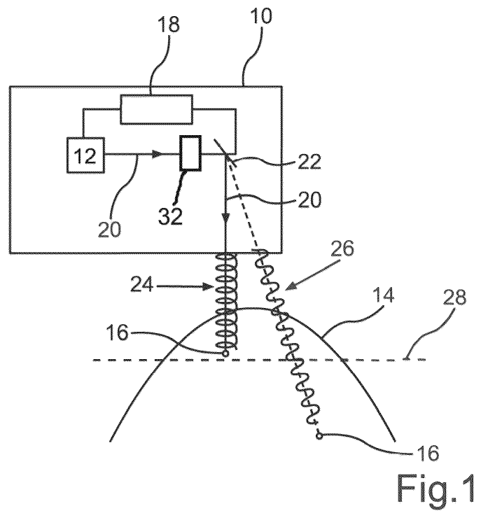

FIG. 1 shows a schematic representation of a treatment apparatus 10 with an ophthalmological laser 12 for generating a laser-induced optical refractive index change. For example, it can be performed for correcting a visual disorder in a cornea 14.

For the laser-induced refractive index change, irradiation positions 16 in the cornea 14 can be provided by a control device 18 for generating a predetermined pattern, in particular in the form of control data, such that the laser 12 emits pulsed laser pulses in a pattern predefined by the irradiation positions 16 into the cornea 14, to change the refractive index in the respective irradiation positions 16. For illustration, only two irradiation positions 16 are represented in this figure, which are in different corneal depths, wherein multiple further irradiation positions can be provided in other corneal depths and/or with other radial position. Furthermore, the control device 18 is illustrated as part of the treatment apparatus 10 in this figure, wherein an external control device can also be provided, for example as part of a planning device.

Furthermore, FIG. 1 shows that the laser beam 20 generated by the laser 12 can be deflected towards the cornea 14 by a beam deflection device 22 such as for example a rotation scanner, to access the irradiation positions 16. The beam deflection device 22 can also be controlled by the control device 18.

In particular, the ophthalmological laser 12 can be a femtosecond laser, which is formed to emit laser pulses in a wavelength range between 300 nanometers and 1400 nanometers, in particular between 700 nanometers and 1200 nanometers, at a respective pulse duration between 1 femtosecond and 1 nanosecond, in particular between 10 femtoseconds and 10 picoseconds, and a repetition frequency of greater than 10 kilohertz, in particular between 100 kilohertz and 100 megahertz.

Furthermore, the treatment apparatus 10 may comprise a polarization device 32 that may be configured to change or adjust a polarization property of the laser beam 20. The polarization device 32 may be arranged in a beam path of the laser beam 20, whereby the position shown in FIG. 1 is to be understood only as an example. The polarization device 32 may comprise a polarizer that may be configured to polarize the laser beam circularly 24 or linearly 26. Alternatively or additionally, waveplates may be provided by which the polarization property may also be set or changed. Furthermore, the polarization device 32 may be configured to change the polarization property of the laser beam 20 by a control signal of the control device, so that it is possible to switch between linear an circular polarization. For example, the polarization device 32 may include an electric motor that sets the polarization property in dependence of the control signal.

In addition, the control device 18 optionally comprises a storage device (not illustrated) for at least temporary storage of at least one control dataset, wherein the control dataset or datasets include(s) control data for positioning and/or for focusing individual laser pulses in the cornea 14. Furthermore, the control device 18 can have access to collagen fiber data, in which an orientation of collagen fibers to be expected in the cornea 14 is stored. The collagen fiber data can, for example, be predetermined by a histological analysis of multiple corneas and/or be determined by measurements of the cornea 14 before the treatment, in particular by microscopic diagnostic measurements.

When performing the laser-induced optical refractive index changes the polarization property of the laser beam 20 may be adapted according to the orientation of the collagen fibers that is provided by the collagen fiber data. In particular, the planned treatment positions 16 may be matched to the collagen fiber data by the control device 18, to obtain the orientation of the collagen fibers to be expected in the respective treatment position 16. The control device 18 then may control the polarization device 32 according the desired polarization property provided by the expected orientation of the collagen fibers.

In particular, it can be assumed that the orientation of the collagen fibers above a preset corneal depth 28 is diffuse and the laser beam 20 may be circularly polarized 24 above that preset corneal depth 28, to obtain an increased refractive index change effect. Thus, the control device 18 may control the polarization device 32 in these areas to provide a circularly polarized laser beam 24.

However, below the preset corneal depth 28, the orientation of the collagen fibers can be orderly, in particular the collagen fibers can be parallel to each other, such that a linear polarization 26 results in an improved refractive index change effect. Herein, the preset corneal depth 28 can be preset by the collagen fiber data. Thus, the control device 18 may control the polarization device 32 in these areas to provide a linearly polarized laser beam 26.

Furthermore, the polarization property can be adapted depending on a radial position in the cornea 14. In particular to provide a polarization angle for the linearly polarized laser beam 26.

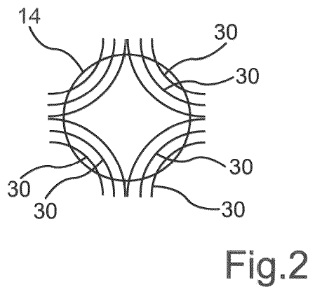

In FIG. 2, a schematic representation of a plane of the cornea 14 is shown, which is in particular below the preset corneal depth 28, in which the orientation of the collagen fibers is orderly. Herein, a progression of the collagen fibers 30 can be assumed to be hyperbolic, wherein multiple quadrants can be point-symmetric to each other. Herein, the polarization angle of the linearly polarized laser beam 26 can be adapted such that the polarization direction coincides with the orientation of the respective collagen fiber 30.

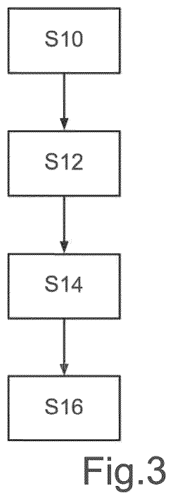

In FIG. 3, a schematic method diagram for providing control data for an ophthalmological laser 12 of a treatment apparatus 10 for the laser-induced refractive index change is illustrated.

In a step S10, treatment positions in a cornea 14, which are intended for the change by the laser-induced refractive index change, can be determined.

In a step S12, an orientation of collagen fibers 30 to be expected in the cornea 14 in the respective treatment positions 16 can be determined based on predetermined collagen fiber data of the cornea 14.

In a step S14, a polarization property of a laser beam 20 of the laser 12 can be set based on the orientation of the collagen fibers 30 to be expected for a respective treatment position 16, wherein the polarization property can in particular be determined based on a radial position and/or a depth of the treatment position 16 in the cornea 14. Herein, a circularly polarized laser beam 24 and/or a linearly polarized laser beam 26 can be adjusted as the polarization property.

Finally, control data can be provided in a step S16, which includes the set polarization property for the respective treatment position.

Overall, the examples show how an improved refractive index change effect can be provided by the use of different polarizations in a cornea.

1. A method for providing control data for an ophthalmological laser of a treatment apparatus for the laser-induced refractive index change, wherein the method comprises the following steps performed by a control device:
determining treatment positions in a cornea of an eye, which are intended for correction by the laser-induced refractive index change;
determining an orientation of collagen fibers to be expected in the cornea in the respective treatment positions based on collagen fiber data of the cornea;
setting a polarization property of a laser beam of the laser based on the orientation of the collagen fibers to be expected for the respective treatment positions;
providing the control data, which includes the set polarization property for the respective treatment positions.

2. The method according to claim 1, wherein the collagen fiber data includes a radial position and/or a depth of the treatment position in the cornea.

3. The method according to claim 1, wherein the polarization property is set to a circularly polarized laser beam and/or a linearly polarized laser beam comprising a polarization angle.

4. The method according to claim 1, wherein the collagen fiber data is predetermined by a histological analysis of multiple corneas.

5. The method according to claim 1, wherein the collagen fiber data is predetermined by measurements of the cornea of the eye.

6. The method according to claim 1, wherein the orientation of the collagen fibers is assumed to be diffuse above a preset corneal depth, and the orientation of the collagen fibers can be parallel below the preset corneal depth, and wherein a circular polarization is set as the polarization property of the laser beam for treatment positions above the preset corneal depth and a linear polarization below the preset corneal depth.

7. The method according to claim 6, wherein a progression of the collagen fibers below the preset corneal depth is assumed to be hyperbolic viewed in a radial direction, and wherein a polarization angle of the linear polarization is adjusted depending on the hyperbolic progression as the polarization property.

8. A control device, which is configured to perform a method according to claim 1.

9. (canceled)

10. A computer-readable medium for storing a computer program, the computer program comprising commands which cause a control device to execute the method according to claim 1.

11. A treatment apparatus comprising at least one ophthalmologic laser for the laser-induced refractive index change of a cornea of a human or animal eye, wherein the treatment apparatus further comprises a control device and a polarization device, wherein the polarization device is configured to adjust a polarization property of a laser beam of the ophthalmologic laser, wherein the control device is configured to determine an expected orientation of collagen fibers in the cornea at predetermined treatment positions in the cornea of the eye that are intended to be changed by the laser-induced refractive index change on the basis of predetermined collagen fiber data of the cornea, and the polarization device is configured to adjust the polarization property depending on the expected orientation of the collagen fibers at the respective treatment position during irradiation with the ophthalmologic laser.

12. The treatment apparatus according to claim 11, wherein the polarization device comprises one or more waveplates and/or one or more rotatable polarizers.
